(19) United States
(12) Patent Application Publication
(10) Pub. No.: US 2024/0034982 A1
(43) Pub. Date: Feb. 1, 2024

(54) Automated Cell Culture Device

(71) Applicant: MEDIGEN BIOTECHNOLOGY CORP., Taipei City (TW)

(72) Inventors: Hsunlang Chang, Taipei City (TW); Chiehliang Lin, Taipei City (TW); Chihya Yang, Taipei City (TW); Chunhsien Liu, Taipei City (TW)

(21) Appl. No.: 18/258,451

(22) PCT Filed: May 25, 2021
(86) PCT No.: PCT/CN2021/095630

§ 371 Date: Jun. 20, 2023

(30) Foreign Application Priority Data
Dec. 23, 2020 (CN) 202011546306.9

(51) Int. Cl. C12M 1/36; C12M 1/00; C12M 3/00

(52) U.S. Cl. CPC C12M 41/48; C12M 23/14; C12M 23/48; C12M 29/20

(57) ABSTRACT

An automated cell culture device includes a main body and a clamping member. The main body encloses a receiving space and has a platform on a top face thereof. The platform has a sliding track extending along a sliding direction. Two ends of the platform along the sliding direction are defined as a first end and a second end respectively. The platform is adapted for support a culture bag lying thereon. The culture bag has a fluid inlet at an end thereof closer to the first end of the platform. The clamping member is slidably disposed on the sliding track and is adapted for clamping and pressing the culture bag to avoid fluid in the culture bag from flowing toward an end of the culture bag closer to the second end of the platform. The clamping member is slidable on the culture bag along the sliding direction.

### BACKGROUND OF THE INVENTION

### Field of the Invention

The present invention relates to a cell culture device.

### Description of the Prior Art

Nowadays, gene therapy or cell therapy often replaces chemical therapy for cancer or other diseases. For example, the immune cell therapy uses the patient's own immune cells to kill cancer cells more efficiently and may not decrease the immunity of the patient. Also, the side effect can be minimized.

The cells of the patient or the donor have to be cultured in vitro, and the cells are further injected into the patient's body. The cell culture is usually proceeded in a soft culture bag. New culture fluid is injected into the culture bag as the cells increase. Besides, the culture fluid has to be arranged in a laboratory environment in specific temperature and humidity.

To acquire the number of cells, the culture fluid in the culture bag is manually extracted for calculation several times. And then, the appropriate amount of culture fluid is injected into the culture bag according to the number of cells. In the whole process of cell culture, this step has to be repeated several times so as to increase the labor cost. In addition, the culture fluid may be contaminated, and the deviation caused by manual operation is inevitable. As a result, the cost of cell culture is too high and unaffordable for patients. Also, the quality of cells is not stable.

The conventional cell culture devices such as the WAVE Bioreactor by Cytiva use a culture bag in a specific size. During the early stage of cell culture, the number of cells and the amount of the culture fluid are low, the cells are dispersed in the whole culture bag to be difficult to interact with other cells. Thus, the efficiency of cell culture is low. To solve this problem, culture bags in different sizes are used in different stages of cell culture. However, the risk of contamination is increased, and the cell culture is impossible to be proceeded automatically.

### SUMMARY OF THE INVENTION

The main object of the present invention is to provide a cell culture device which is able to gather the culture fluid to stabilize the quality of cell culture so that the cell culture device is beneficial to automated culture.

To achieve the above and other objects, the automated cell culture device of the present invention includes a main body and a clamping member.

T main body encloses a receiving space and has a platform on a top face thereof. The platform has at least one sliding track extending along a sliding direction. Two ends of the platform along the sliding direction are defined as a first end and a second end respectively. The platform is adapted for support a culture bag lying thereon. The culture bag has at least one fluid inlet located at an end thereof closer to the first end of the platform. The clamping member is slidably disposed on the at least one sliding track. The clamping member is adapted for clamping and pressing the culture bag to avoid fluid in the culture bag from flowing toward an end of the culture bag closer to the second end of the platform. The clamping member is slidable on the culture bag along the sliding direction.

Therefore, the cell culture device of the present invention is able to gather the culture fluid during the gradual injections of culture fluid. Thus, the quality and efficiency of cell culture can be maintained because the culture fluid is prevented from dispersing in the whole culture bag. In addition, the present invention can cooperate with the culture fluid injection device so as to achieve the automated culture process.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of the present invention;

FIG. 2 is an illustration of use of the present invention;

FIG. 3 is a profile of the present invention;

FIG. 4 to FIG. 7 are profiles of the present invention during operation;

FIG. 8 is a comparison graph of the present invention and the prior art.

### DETAILED DESCRIPTION OF THE PREFERRED EMBODIMENTS

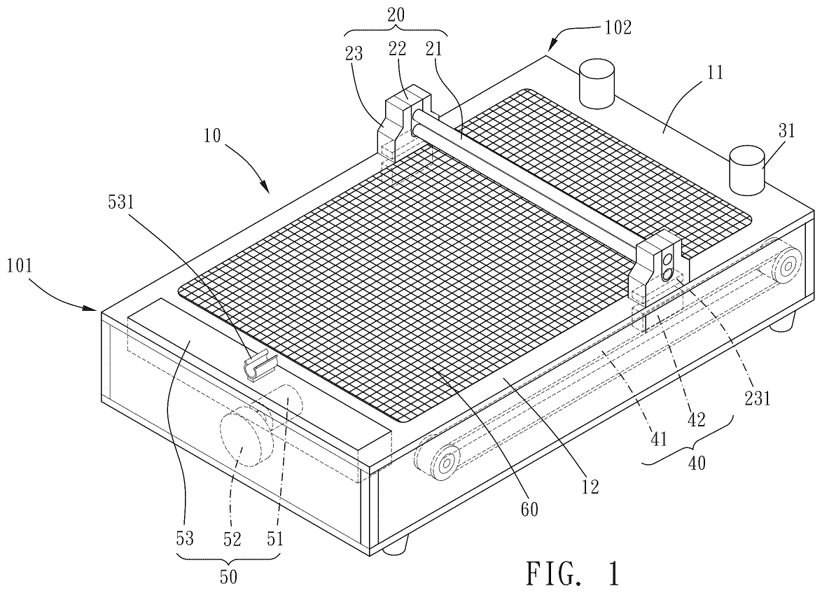

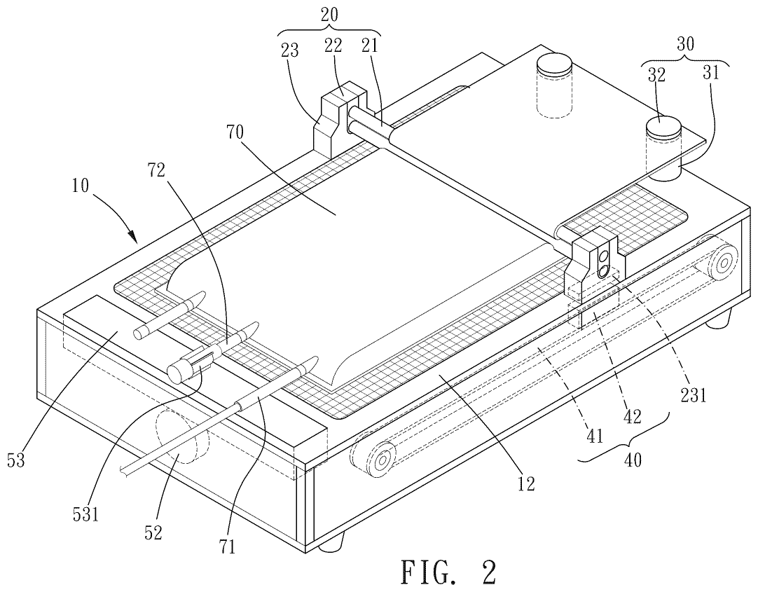

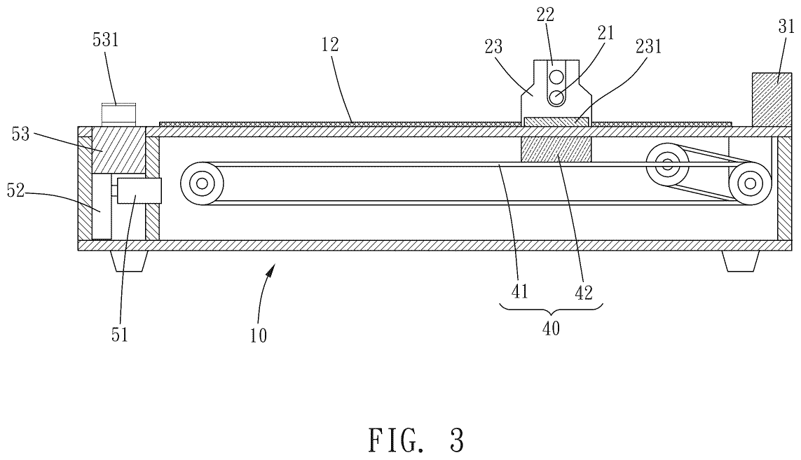

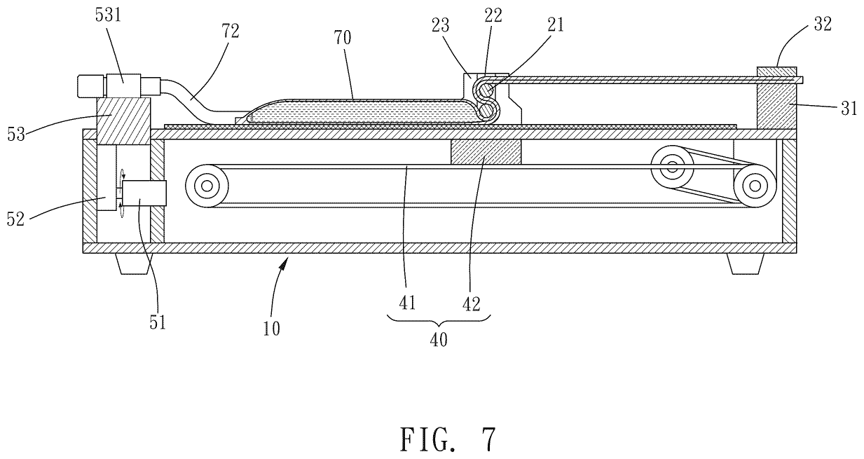

Please refer to FIG. 1 to FIG. 7, the automated cell culture device of the present invention includes a main body 10 and a clamping member 20, and further includes at least one pulling mechanism, a movement driving mechanism 40, and a pushing mechanism 50 optionally.

The main body 10 encloses a receiving space and is formed with a platform 11 on a top face thereof. A sliding track 12 is configured at each of two sides of the platform 11. Each of the sliding tracks 12 extends along a sliding direction. The two ends of the platform 11 along the sliding direction are defined as a first end 101 and a second end 102. The platform 11 is adapted for supporting a culture bag 70 lying thereon. The culture bag 70 is adapted for receiving the culture fluid and the cells for culture. The culture bag 70 has at least one fluid inlet 71 for the injection of the culture fluid, and the fluid inlet 71 is located at an end of the culture bag 70 closer to the first end 101 and is adapted for connecting to a culture fluid source, such as a peristaltic pump. Preferably, a ventilation member 60 having a bumpy surface is disposed on the platform 11 and has convex structures and concave structures. The ventilation member 60 is adapted for being sandwiched between the platform 11 and the culture bag 70 so as to facilitate the breathing of the bottom of the culture bag 70. For example, the ventilation member 60 can be a metal mesh, a pegboard, or other board-like object having the similar structure. When a pegboard is used as the ventilation member, the top surface of the pegboard can be regarded as a convex structure, and the holes of the pegboard can be regarded as a concave structure.

The clamping member 20 has two opposite ends slidably disposed on the two sliding tracks 12 respectively. The clamping member 20 is adapted for clamping and pressing the culture bag 70 to prevent the fluid in the culture bag 70 from flowing toward the end of the culture bag 70 closer to the second end 102 of the platform 11. For example, the clamping member can directly press the culture bag toward the platform, or clamp the culture bag in other ways to block the culture fluid. The clamping member 20 is slidable on the culture bag 70 along the sliding direction.

The pulling mechanism is disposed on at least the second end 102 of the platform 11 in order to hold the end of the culture bag 70 closer to the second end 102 of the platform 11. Preferably, the at least one pulling mechanism is plural and is also disposed on the first end of the platform to hold the end of the culture bag closer to the first end of the platform. Specifically, the at least one pulling mechanism includes at least two gripping members 30 disposed on the main body 10 at two opposite sides of the main body 10 near the two sliding tracks 12 in order to grip two opposite sides of the end of the culture bag 70 closer to the second end 102 of the platform 11. The pulling structure disposed on the first end of the platform is arranged similarly. More specifically, in the present embodiment, each of the gripping members 30 has two magnetic pieces 31,32. One of the magnetic pieces 31 is fixedly disposed on the platform 11, and the other one of the magnetic pieces 32 is detachably attached to the magnetic piece 31 fixedly disposed on the platform 11 so as to clamp a corner of the culture bag 70 therebetween. However, the gripping member can have other structures, such as conventional clips. Preferably, the two magnetic pieces 31,32 are higher than the clamping member so that the end of the culture bag closer to the second end of the platform is higher than other portions of the culture bag. Thus, the culture fluid is avoided to flow toward the end of the culture bag closer to the second end by the gravity and the capillary action.

In the present embodiment, the clamping member 20 has two cross bars 21, two connection member 22, and two sliding members 23. Each of the two cross bars 21 is horizontally arranged and is perpendicular to the sliding direction. The two cross bars 21 are spacedly arranged. One of the cross bars 21 is closer to the platform 11 than the other one cross bar 21. That is, the two cross bars 21 have various heights from the platform 11. Two opposite ends of each of the cross bars 21 are fixedly connected to the two connection members 22. The two connection members 22 are detachably disposed on the two sliding members 23 respectively. The two sliding members 23 are slidably disposed on the two sliding tracks 12 respectively. For example, each of the sliding members 23 can have a groove for the connection member 22 to insert in.

The movement driving mechanism 40 is received in the receiving space of the main body 10. In the present embodiment, the movement driving mechanism 40 includes a belt assembly 41 and two magnetic portions 42. The two magnetic portions 42 are disposed on the belt assembly 41 to be movable along the sliding direction due to the belt assembly 41. Specifically, each of the sliding members 23 has a magnetic member 231. The magnetic members 231 of the sliding members 23 are magnetically attached to the two magnetic portions 42 respectively so that the two sliding members 23 can be driven to move along the sliding direction by the belt assembly 41. Thus, the sliding track doesn't have to penetrate the platform so that the receiving space of the main body is isolated from the exterior. Therefore, the components in the receiving space are prevented from rusting.

The pushing mechanism 50 is received in the receiving space of the main body 10 and is located at the first end 101 of the platform. The pushing mechanism 50 is liftable to reciprocally lift and lower the end of the culture bag 70 closer to the first end 101. Thus, the culture fluid in the culture bag 70 can be slightly shaken to be mixed well. In the preset embodiment, the pushing mechanism 50 includes a rotation device 51, an eccentric cam 52, and a pushing plate 53. The rotation device 51 can be a rotation axle which is driven to rotate by motors or others. The eccentric cam 52 is eccentrically disposed on the rotation device 51 and is rotated eccentrically by the rotation device 51. The top edge of the eccentric cam 52 is adapted for abutting against the pushing plate 53. The pushing plate 53 is adapted for abutting against the culture bag 70. When the eccentric cam 52 rotates eccentrically, the height of the eccentric cam 52 changes so that the pushing plate 53 is reciprocally lifted and lowered by the eccentric cam 52. However, other conventional lift mechanisms are acceptable too.

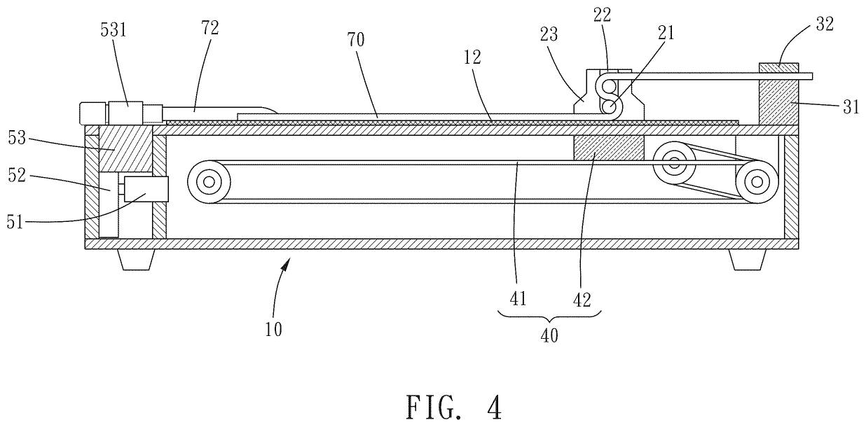

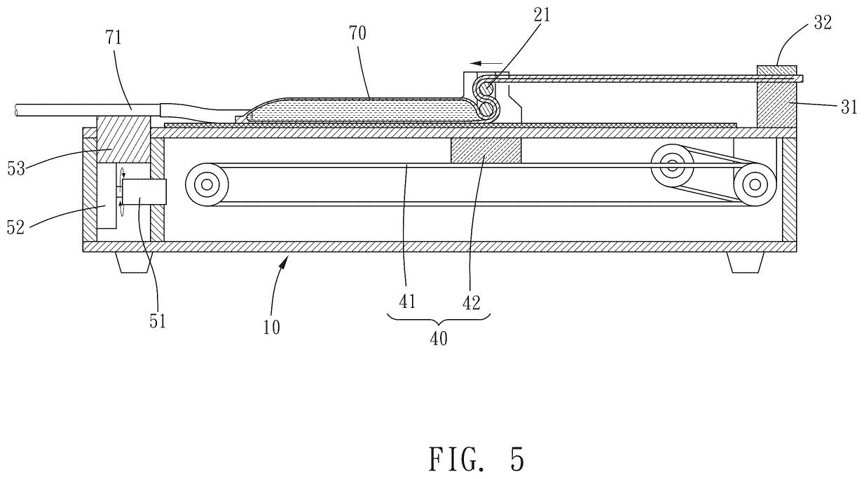

In practice, as shown in FIG. 4, the end of the culture bag 71 opposite to the fluid inlet 71 is inserted through the gap between the two cross bars 21 toward the first end 101 and lies on the upper cross bar 21 to further extend toward the second end 102 and to be positioned at the second end 102 by the two gripping members 30. Thus, the culture bag 70 is wound on the two cross bars 21 to be pressed and clamped, and the end of the culture bag 70 corresponding to the second end 102 becomes higher than the other end of the culture bag 70 so that the culture fluid in the culture bag 70 is prevented from flowing toward the second end 102. In the present embodiment, the “automated” cell culture device means that the clamping member moves automatically. However, the clamping member can be adjusted manually in other embodiments. Besides, the end of the culture bag 70 corresponding to the first end 101 has a fixation portion 72, such as a sampling tube. The pushing plate 53 has a corresponding fixation member 531. The fixation portion 72 can be connected to the fixation member 531 to position the two ends of the culture bag 70 on the main body 10. Thus, when the clamping member 20 moves along the sliding direction, the culture fluid in the culture bag 70 is pushed toward the fluid inlet 71 to prevent the culture fluid from dispersing in the whole culture bag 70. Thus, the cells can contact enough amount of cells from the beginning, as shown in FIG. 5. In the present embodiment, the fixation member 531 is elastic and deformable clip to grasp the tube of the fixation portion 72. However, the fixation member can be a pinchcock, a magnetic clip, or other types of clips in other embodiments.

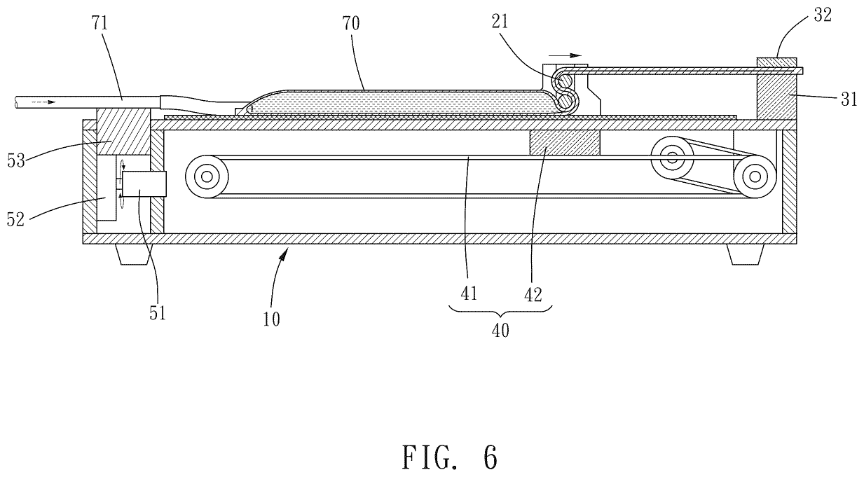

During the process of cell culture, new culture fluid is gradually injected into the culture bag 70. Thus, the clamping member 20 is moved continually toward the second end 102 or moved in several times, as shown in FIG. 6, in order to enlarge the space in the culture bag. The timing, the speed, and the distance of movement can be adjusted and controlled according to the necessary amount of culture fluid which is referred to the desired cell amount.

Besides, as shown in FIG. 5 to FIG. 7, the pushing mechanism 50 can lift and lower the end of the culture bag 70 closer to the first end 101 reciprocally to slightly shake the culture fluid in the culture bag 70. Thus, the cells can be distributed evenly in the culture fluid during the injection of culture fluid.

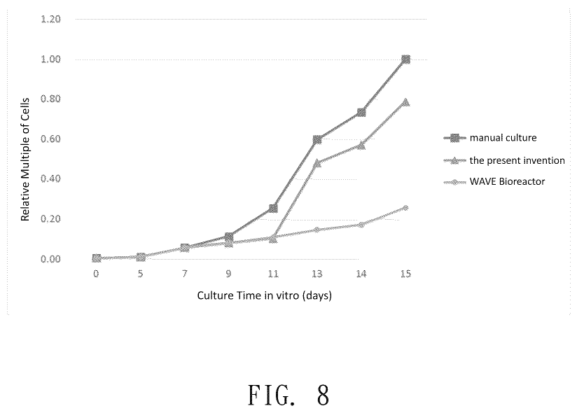

Please refer to FIG. 8 for the performance of the present invention. The natural killer cells separated from the peripheral blood sample of a single subject are cultured in three devices or ways including (i) the cell culture device of the present invention, (ii) the conventional cell culture device “WAVE Bioreator”, and (iii) manual cell culture without cell culture device. The peripheral blood sample is taken from the arm of the subject following the plans recognized by the Research Ethic Committee. The original cell amount, the culture medium, and the culture time of the three ways are the same. As shown in FIG. 8, the multiple of the cells in the present invention reaches 80% that of manual cell culture in 15 days. That is, the cell culture device is able to replace manual cell culture to economize manpower. Besides, FIG. 8 also shows that the multiple of cells in the conventional WAVE Bioreactor is only 26% that of manual cell culture in 15 days to be significantly lower than the present invention. Thus, the present invention can certainly improve the efficiency of cell culture device.

The present invention can further connect and cooperate with the culture fluid source and the detection mechanism of temperature, gas, pH, and humidity in order to calculate the frequency, the speed, and amount of injection. And then, the timing of movement of the clamping member and the moving speed of the clamping member can be set according to the data, and the pushing mechanism is driven at the appropriate timing in the appropriate frequency to mix the culture fluid and the cells.

Thus, the process of culture can be controlled automatically without manual injection and shaking of culture fluid, and the culture fluid can be gathered to make the cells multiple in the stable condition. Thus, the efficiency and yield of cell culture can be increased, and the manpower can be saved so as to diminish the deviation caused by manual operation.

Therefore, the present invention can economize manpower to maintain the sufficient quality of cell culture so as to decrease the cost. Thus, the cell therapy becomes cheaper to be affordable.

Embodiments are shown below that the present invention discloses:

1. An automated cell culture device, including:
a main body, enclosing a receiving space and having a platform on a top face thereof, the platform having at least one sliding track extending along a sliding direction, two ends of the platform along the sliding direction being defined as a first end and a second end respectively, the platform being adapted for support a culture bag lying thereon, wherein the culture bag has at least one fluid inlet located at an end thereof closer to the first end of the platform;
a clamping member, slidably disposed on the at least one sliding track, the clamping member being adapted for clamping and pressing the culture bag to avoid fluid in the culture bag from flowing toward an end of the culture bag closer to the second end of the platform, the clamping member being slidable on the culture bag along the sliding direction.

2. The automated cell culture device of embodiment 1, further including at least one pulling mechanism at least disposed on the second end of the platform, the at least one pulling mechanism being adapted for pulling and retaining the end of the culture bag closer to the second end of the platform.

3. The automated cell culture device of embodiment 2, wherein the at least one pulling mechanism includes at least two gripping members to hold two sides of the end of the culture bag closer to the second end of the platform respectively.

4. The automated cell culture device of embodiment 3, wherein each of the gripping members includes two magnetic pieces, one of the magnetic pieces is fixedly disposed on the platform, the other one of the magnetic pieces is detachably attached to the magnetic piece fixedly disposed on the platform so as to clamping a corner of the culture bag between the two magnetic pieces.

5. The automated cell culture device of embodiment 1, wherein the clamping member includes two cross bars arranged spacedly, each of the cross bars is perpendicular to the sliding direction, one of the cross bars is closer to the platform than the other cross bar is.

6. The automated cell culture device of embodiment 5, wherein one said sliding track is arranged at each of two sides of the platform, the clamping member includes two connection members and two sliding members, two ends of each of the cross bars are connected to the two connection members respectively, the two connection members are detachably disposed on the two sliding members respectively, the two sliding members are slidably disposed on the two sliding tracks respectively.

7. The automated cell culture device of embodiment 6, further including a movement driving mechanism configured in the receiving space of the main body, the movement driving mechanism including a belt assembly and two magnetic portions, the two magnetic portions being arranged on the belt assembly and being driven by the belt assembly to move along the sliding direction, each of the sliding members having a magnetic member, the magnetic members of the two sliding members being attached magnetically to the two magnetic portions respectively so that the two sliding members are driven to move along the sliding direction by the belt assembly.

8. The automated cell culture device of embodiment 1, further including a pushing mechanism arranged in the receiving space of the main body at the first end of the main body, the pushing mechanism being liftable to lift and lower the end of the culture bag closer to the first end reciprocatedly.

9. The automated cell culture device of embodiment 8, wherein the pushing mechanism includes a rotation device, an eccentric cam, and a pushing plate, the eccentric cam is eccentrically disposed on the rotation device to eccentrically rotate with the rotation device, an edge of the eccentric cam is adapted for abutting against the pushing plate, the pushing plate is adapted for abutting against the culture bag, the pushing plate is lifted and lowered reciprocatedly by the eccentric cam when the eccentric cam rotates eccentrically.

10. The automated cell culture device of embodiment 1, further including a ventilation member disposed on the platform and having a bumpy surface, the ventilation member being adapted for being sandwiched between the platform and the culture bag so as to facilitate breathing of a bottom of the culture bag.

What is claimed is:

1. An automated cell culture device, including:
a main body, enclosing a receiving space and having a platform on a top face thereof, the platform having at least one sliding track extending along a sliding direction, two ends of the platform along the sliding direction being defined as a first end and a second end respectively, the platform being adapted for support a culture bag lying thereon, wherein the culture bag has at least one fluid inlet located at an end thereof closer to the first end of the platform;
a clamping member, slidably disposed on the at least one sliding track, the clamping member being adapted for clamping and pressing the culture bag to avoid fluid in the culture bag from flowing toward an end of the culture bag closer to the second end of the platform, the clamping member being slidable on the culture bag along the sliding direction.

2. The automated cell culture device of claim 1, further including at least one pulling mechanism at least disposed on the second end of the platform, the at least one pulling mechanism being adapted for pulling and retaining the end of the culture bag closer to the second end of the platform.

3. The automated cell culture device of claim 2, wherein the at least one pulling mechanism includes at least two gripping members to hold two sides of the end of the culture bag closer to the second end of the platform respectively.

4. The automated cell culture device of claim 3, wherein each of the gripping members includes two magnetic pieces, one of the magnetic pieces is fixedly disposed on the platform, the other one of the magnetic pieces is detachably attached to the magnetic piece fixedly disposed on the platform so as to clamping a corner of the culture bag between the two magnetic pieces.

5. The automated cell culture device of claim 1, wherein the clamping member includes two cross bars arranged spacedly, each of the cross bars is perpendicular to the sliding direction, one of the cross bars is closer to the platform than the other cross bar is.

6. The automated cell culture device of claim 5, wherein one said sliding track is arranged at each of two sides of the platform, the clamping member includes two connection members and two sliding members, two ends of each of the cross bars are connected to the two connection members respectively, the two connection members are detachably disposed on the two sliding members respectively, the two sliding members are slidably disposed on the two sliding tracks respectively.

7. The automated cell culture device of claim 6, further including a movement driving mechanism configured in the receiving space of the main body, the movement driving mechanism including a belt assembly and two magnetic portions, the two magnetic portions being arranged on the belt assembly and being driven by the belt assembly to move along the sliding direction, each of the sliding members having a magnetic member, the magnetic members of the two sliding members being attached magnetically to the two magnetic portions respectively so that the two sliding members are driven to move along the sliding direction by the belt assembly.

8. The automated cell culture device of claim 1, further including a pushing mechanism arranged in the receiving space of the main body at the first end of the main body, the pushing mechanism being liftable to lift and lower the end of the culture bag closer to the first end reciprocatedly.

9. The automated cell culture device of claim 8, wherein the pushing mechanism includes a rotation device, an eccentric cam, and a pushing plate, the eccentric cam is eccentrically disposed on the rotation device to eccentrically rotate with the rotation device, an edge of the eccentric cam is adapted for abutting against the pushing plate, the pushing plate is adapted for abutting against the culture bag, the pushing plate is lifted and lowered reciprocatedly by the eccentric cam when the eccentric cam rotates eccentrically.

10. The automated cell culture device of claim 1, further including a ventilation member disposed on the platform and having a bumpy surface, the ventilation member being adapted for being sandwiched between the platform and the culture bag so as to facilitate breathing of a bottom of the culture bag.
